(19) United States
(12) Patent Application Publication
(10) Pub. No.: US 2021/0236676 A1
(43) Pub. Date: Aug. 5, 2021

(54) APPARATUS FOR PROCESSING MEDICAL INSTRUMENTS

(71) Applicant: Cybersterile LLC, Miami, FL (US)

(72) Inventors: Diego Araujo Dalla Bona, Miami, FL (US); Ali Panahi, Plantation, FL (US); Rafael Paschoalotto, Kissimmee, FL (US)

(21) Appl. No.: 17/103,970

(22) Filed: Nov. 25, 2020

Related U.S. Application Data
(60) Provisional application No. 62/967,659, filed on Jan. 30, 2020.

(51) Int. Cl. A61L 2/24; B65B 55/02; B65B 43/46; B65B 65/00; A61L 2/06

(52) U.S. Cl. CPC A61L 2/24; B65B 55/025; B65B 43/46; B65B 65/003; A61L 2/06; A61L 2202/24; A61L 2202/121; A61L 2202/17; A61L 2202/18; A61L 2202/14; A61L 2202/122

(57) ABSTRACT

An apparatus for processing medical instruments includes an enclosure having an entrance door and an exit door. The enclosure is arranged so that a cassette containing dirty instruments is deposited into the enclosure through the entrance door, the dirty instruments are cleaned, sterilized, and packaged inside the enclosure, and the clean, sterilized, packaged instruments are removed from the enclosure through the exit door. The enclosure includes a plurality of stations therein, such as a jet washing and drying station, a packaging station, and an autoclave station. Each station has sidewalls and a bottom surface, and includes at least one slot that is sized and shaped to hold only one cassette. Each slot in each station has a wire frame so that the cassette, when positioned in the slot, does not contact the sidewalls or bottom surface of the station. A robotic arm automatically moves the instrument cassettes between stations.

### CROSS-REFERENCE TO RELATED APPLICATIONS

This application claims priority from U.S. Provisional Patent Application No. 62/967,659, filed Jan. 30, 2020, entitled “Fully Automated Machine to Clean Dry Pack and Sterilize Medical and Dental Instruments,” which is hereby incorporated by reference herein for all purposes.

### BACKGROUND

### Field of the Art

This disclosure relates to an automated apparatus for processing medical instruments. In particular, the apparatus is configured for automatically cleaning, drying, packaging, and sterilizing reusable medical instruments.

### Discussion of the State of the Art

Reusable medical devices are devices that health care providers can reuse to diagnose and treat multiple patients. Examples of reusable medical devices include surgical forceps, restorative spatulas, dental mirrors, periodontal scalers, endoscopes, and stethoscopes

When used on patients, reusable instruments become soiled and contaminated with microorganisms and blood. To avoid any risk of infection by a contaminated instrument, reusable instruments undergo “reprocessing,” which is a detailed multistep process to clean, dry, pack, and sterilize the instruments. Adequate reprocessing of reusable medical instruments is vital to protecting patient safety. Inadequate cleaning between patient uses can result in the retention of blood, tissue, and other biological debris (soil) in certain types of reusable instruments. This debris can allow microbes to survive the subsequent disinfection or sterilization process, which could then lead to Health Care-Associated Infections (HAIs).

Inadequate cleaning and the presence of debris can compromise the sterilization of reusable instruments. Cleaning should always occur before sterilization. Currently, there are a few solutions for management of reusable medical and dental instruments. Some of these solutions attempt to clean (e.g., manually brush) and dry, or to pack, or to sterilize, but these solutions fail to meet the needs of the dental and medical offices because they have to rely on people/employers. In addition, sometimes these solutions do not follow the correct protocol established by the CDC. Also, there is a lack of verification and process reports. Other solutions attempt to automatically clean and dry reusable instruments (e.g., ultrasonic cleaners, instrument washers, washer-disinfectors, and the like), but these solutions are similarly unable to meet the needs of the industry because they still require a user to pack, load, and unload the autoclave machine. A typical sterilization center in a dental office, for example, may take up an entire room and require a user to move the instruments from one module to the next. This results in user error, inadequate cleaning and sterilizing, and a lot of wasted space and time.

Another common problem involves overloading the autoclave. Overloading results in inadequate sterilization. In addition, overloading may cause instrument packs to retain moisture and come out of the autoclave wet. As such, there is a need for solution that adequately and efficiently cleans, dries, packages, and sterilizes instruments for re-use.

### SUMMARY

It is desirable to have a fully automated system that will manage reprocessing of reusable instruments in dental and medical offices or hospitals. Such a system would increase efficiency, reduce work time, and decrease the risk of injury to a user during management of infected instruments. Furthermore, it is desirable to have a system that will have sensors to identify any failure of the process. Still further, it is desirable to have a system that will automatically follow all recommendations from the CDC about how to manage reusable instrument in medical and dental facilities, resulting in increased safety to employers and to patients. The disclosed system advantageously fills these needs and addresses the aforementioned deficiencies by providing a compliant, fully automated system to manage reusable instruments.

One advantage of the disclosed system is that it is compact and yet it performs all the functions necessary to clean, dry, package, and sterilize medical instruments. In particular, the system is about the size of a cabinet unit typically found in a medical or dental office.

Another advantage of the system is that it is fully automated. A user only needs to pack the dirty instruments in a cassette, place the cassette into the machine, and retrieve the cleaned, sterilized, packaged cassette at the end of the cycle.

In one embodiment, the present invention includes an apparatus for processing medical instruments. The apparatus includes an enclosure having an entrance door and an exit door. The entrance door and exit door may open and close automatically. The enclosure is arranged so that a cassette containing dirty instruments is deposited into the enclosure through the entrance door, the dirty instruments are cleaned, packaged, and sterilized inside the enclosure, and the clean, packaged, sterilized instruments are removed from the enclosure through the exit door. The enclosure includes a plurality of stations therein. The stations may be arranged in a linear row. Each station has sidewalls and a bottom surface. Each station has at least one slot that is sized and shaped to hold only one cassette. Each slot in each station includes a wire frame so that the cassette, when positioned in the slot, does not contact the sidewalls or bottom surface of the station. Each station may include at least two or three slots, with each slot being sized and shaped to hold only one cassette.

One of the stations is a jet washing and drying station. The jet washing and drying station may have a door that is configured to be closed during washing and drying, and is further configured to be open before and after the washing and drying. The jet washing and drying station door may be configured to open and close automatically.

Another one of the stations is a packaging station. The packaging station may include a roller of packaging material, an air blower for opening the package before the cassette is positioned in the package, and a heat sealer for sealing the package after the cassette is positioned therein.

Yet another one of the stations is an autoclave station. The autoclave station may include a lid that closes to seal the autoclave station closed during operation of the autoclave station. The autoclave station lid may be configured to open and close automatically.

The jet washing and drying station may be adjacent to the packaging station, and the packaging station may be disposed between the jet washing and drying station and the autoclave station.

The apparatus may further include an ultrasound cleaning station, which may be configured to automatically turn on when the cassette is positioned therein. An instrument cleaning solution reservoir may be coupled to the ultrasound cleaning station. The ultrasound cleaning station may be configured for being coupled to a water source. The apparatus may further include a filter for filtering water that enters the ultrasound cleaning station. The ultrasound cleaning station may include a door that is configured to be closed during cleaning, wherein the door opens to provide access to the ultrasound cleaning station for depositing the cassette into, or removing the cassette from, the ultrasound cleaning station. The ultrasound cleaning station door may be configured to open and close automatically.

The apparatus may further include a holding area in the enclosure, and the holding area may be adjacent to the exit door. Still further, the apparatus may include a receiving area in the enclosure, wherein the receiving area is positioned adjacent to the entrance door so that a user has access to the receiving area through the entrance door in order to position the instrument cassette through the entrance door an into the receiving area. The apparatus may further include a fluid sprayer adapted to be coupled to a pre-cleaning solution for spraying the pre-cleaning solution on the external surfaces of the dirty instruments. The fluid sprayer may be within, or adjacent to, the receiving area.

The apparatus may further include a robotic arm in the enclosure. The robotic arm may be coupled to a linear track positioned above the stations in the enclosure. The robotic arm may be configured to traverse the linear track in the y-direction. The robotic arm may further be configured to move in the z-direction towards and away from the stations in the enclosure. The robotic arm may include a gripper that closes and opens for grasping and releasing the cassettes. Further, a second gripper may be attached to the robotic arm. The apparatus may further include two robotic arms in the enclosure, wherein a first robotic arm is configured for engaging with the cassette containing dirty instruments, and a second robotic arm is configured for engaging with the cassette after the instruments have been cleaned.

The apparatus may further include a camera and a processor configured for automatically determining whether the medical instruments are clean. The camera and processor may be positioned adjacent to the jet washing and drying station in order to determine whether the instruments are clean prior to packaging. The processor may include artificial intelligence capability for determining whether the instruments are clean.

The apparatus may further include a UV light in the enclosure for sterilizing the inside of the enclosure.

In another embodiment, the present invention includes an apparatus for processing medical instruments. The apparatus includes an enclosure having an entrance door and an exit door. The enclosure is arranged so that a cassette containing dirty instruments is deposited into the enclosure through the entrance door, the dirty instruments are cleaned, sterilized, and packaged inside the enclosure, and the clean, sterilized, packaged instruments are removed from the enclosure through the exit door. The enclosure includes a plurality of stations arranged in a linear row. Each station has sidewalls, a bottom surface, and at least one slot that is sized and shaped to hold only one cassette. Each slot in each station includes a wire frame configured to hold the cassette in a position that is spaced apart from the sidewalls and bottom surface of the station so that all sides of the instruments are fluidly accessible through the cassette and the wire frame. The plurality of stations may include an ultrasound cleaning station, a jet washing and drying station, a packaging station, and an autoclave station. A receiving area may be adjacent to the entrance door and a holding area may be adjacent to the exit door.

Other and further aspects, advantages, and features of the disclosed embodiments will become apparent from the ensuing detailed description in view of the accompanying drawings.

### BRIEF DESCRIPTION OF THE DRAWING FIGURES

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 is a perspective view of an apparatus for reprocessing medical instruments, in accordance with an embodiment of the present invention.

FIG. 2 illustrates an entrance door of the apparatus shown in FIG. 1, in accordance with an embodiment of the present invention.

FIG. 3 illustrates an exit door of the apparatus shown in FIG. 1, in accordance with an embodiment of the present invention.

FIG. 4 illustrates a display screen and printer of the apparatus shown in FIG. 1, in accordance with an embodiment of the present invention.

FIG. 5A illustrates instrument sterilization cassettes, in accordance with an embodiment of the present invention.

FIG. 5B illustrates instrument sterilization cassettes, in accordance with an embodiment of the present invention.

FIG. 6A illustrates the interior components of the apparatus shown in FIG. 1, in accordance with an embodiment of the present invention.

FIG. 6B illustrates the interior components of the apparatus shown in FIG. 1, in accordance with an embodiment of the present invention.

FIG. 7A illustrates details of a jet washing and drying station, in accordance with an embodiment of the present invention.

FIG. 7B illustrates details of a jet washing and drying station, in accordance with an embodiment of the present invention.

FIG. 7C illustrates details of a jet washing and drying station, in accordance with an embodiment of the present invention.

FIG. 7D illustrates details of a jet washing and drying station, in accordance with an embodiment of the present invention.

FIG. 8A illustrates details of a packaging station, in accordance with an embodiment of the present invention.

FIG. 8B illustrates details of a packaging station, in accordance with an embodiment of the present invention.

FIG. 8C illustrates details of a packaging station, in accordance with an embodiment of the present invention.

FIG. 8D illustrates details of a packaging station, in accordance with an embodiment of the present invention.

FIG. 8E illustrates details of a packaging station, in accordance with an embodiment of the present invention.

FIG. 8F illustrates details of a packaging station, in accordance with an embodiment of the present invention.

FIG. 8G illustrates details of a packaging station, in accordance with an embodiment of the present invention.

FIG. 9 illustrates a holding area of the apparatus shown in FIG. 1, in accordance with an embodiment of the present invention.

FIG. 10A illustrates details of a robotic arm, in accordance with an embodiment of the present invention.

FIG. 10B illustrates details of a robotic arm, in accordance with an embodiment of the present invention.

FIG. 10C illustrates details of a robotic arm, in accordance with an embodiment of the present invention.

FIG. 10D illustrates details of a robotic arm, in accordance with an embodiment of the present invention.

FIG. 11A illustrates details of a robotic arm, in accordance with another embodiment of the present invention.

FIG. 11B illustrates details of a robotic arm, in accordance with another embodiment of the present invention.

FIG. 11C illustrates details of a robotic arm, in accordance with another embodiment of the present invention.

### DETAILED DESCRIPTION

Disclosed and depicted herein are systems and devices for automatically reprocessing medical instruments. In one embodiment, a compact apparatus for reprocessing medical instruments includes a cleaning station, a drying station, a packaging station, and/or a sterilizing station. The apparatus may further include a storage area for dirty, contaminated instruments that need to be processed, and a storage area for the clean, sterilized instruments that have been processed.

The instruments are disposed in a sterilization cassette and the cassette is automatically moved from one station to the next station with a robotic arm, conveyor belt, or the like. Each station includes at least one slot that is sized and shaped to hold only one sterilization cassette to avoid over-crowding of the cassettes in each station. The apparatus is compact relative to other sterilization centers that are typically found in dental or medical offices. For example, the apparatus is about the size of a standard cabinet unit that is typically present in dental or medical offices.

The invention is described by reference to various elements herein. It should be noted, however, that although the various elements of the inventive apparatus are described separately below, the elements need not necessarily be separate. The various embodiments may be interconnected and may be cut out of a singular block or mold. The variety of different ways of forming an inventive apparatus, in accordance with the disclosure herein, may be varied without departing from the scope of the invention.

Generally, one or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices and parts that are connected to each other need not be in continuous connection with each other, unless expressly specified otherwise. In addition, devices and parts that are connected with each other may be connected directly or indirectly through one or more connection means or intermediaries.

A description of an aspect with several components in connection with each other does not imply that all such components are required. To the contrary, a variety of optional components may be described to illustrate a wide variety of possible embodiments and in order to more fully illustrate one or more embodiments. Similarly, although process steps, method steps, or the like may be described in a sequential order, such processes and methods may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, or method is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Alternate implementations are included within the scope of various embodiments in which, for example, functions may be executed out of order from that shown or discussed, including substantially concurrently or in reverse order, depending on the functionality involved, as would be understood by those having ordinary skill in the art.

The apparatus of the present invention is for processing medical and dental instruments that are positioned inside a sterilization cassette. The apparatus includes an enclosure having an entrance door and an exit door. Within the enclosure are several stations. In one embodiment, the stations include a jet washing and drying station, a packaging station, and an autoclave station. Each station has sidewalls and a bottom surface. A wire frame slot in each station is sized and shaped to hold one cassette in a position that is spaced apart from the sidewalls and bottom surface of the station.

The present invention is directed to a fully automated machine to clean, dry, pack, and sterilize medical and dental instruments. The machine can be of all sizes and can support various sizes and quantities of cassettes of instruments. Also, instruments can be of all types, such as pliers, forceps, scissors, burs, hand pieces, etc.

In one embodiment, the present invention comprises a fully automated machine to clean (e.g., with ultrasound and/or power wash), dry, pack, and heat sterilize medical and dental instruments. More particularly, the invention is a complete automation of the management of reusable instrument in dental facilities, medical facilities, and hospitals. The machine may include a dirty/infection door, where dental and medical assistants may load the machine with cassettes containing dirty reusable instruments. A mechanical/robotic arm may be used to move the cassette through different compartments inside the machine, such as ultrasound cleaning, power washing, drying system, automated packing, and/or heat sterilization. Finally, the robotic arm may transport the cleaned instruments to the clean door on the other side of the machine where the clean instruments inside the cassette will be ready to be used or stored.

The compartments of the machine may include storage for uncleaned cassettes, ultrasonic cleaning, power wash and dry, packaging, autoclave, and/or storage for clean cassettes. A mechanical arm may be used to move around the machine to transport cassettes inside the machine. The machine may have several other compartments and be provided in a variety of different size ranges. For example, depending on the desired size range, some of the compartments may be eliminated for a smaller apparatus, or some of the compartments may be duplicated for a larger apparatus. In one particular example, a user may require an apparatus having an additional autoclave compartment. In another example, a user may require an apparatus having only a power wash and dry compartment. Regardless of the configuration or number of compartments within the apparatus, it is important to note that each compartment includes at least one slot for holding only one sterilization cassette. In this manner, the sterilization cassettes being processed are held in a position that is spaced apart from the sidewalls and bottom surface of the compartment and from other sterilization cassettes, which results in instruments that are more efficiently and effectively cleaned and sterilized.

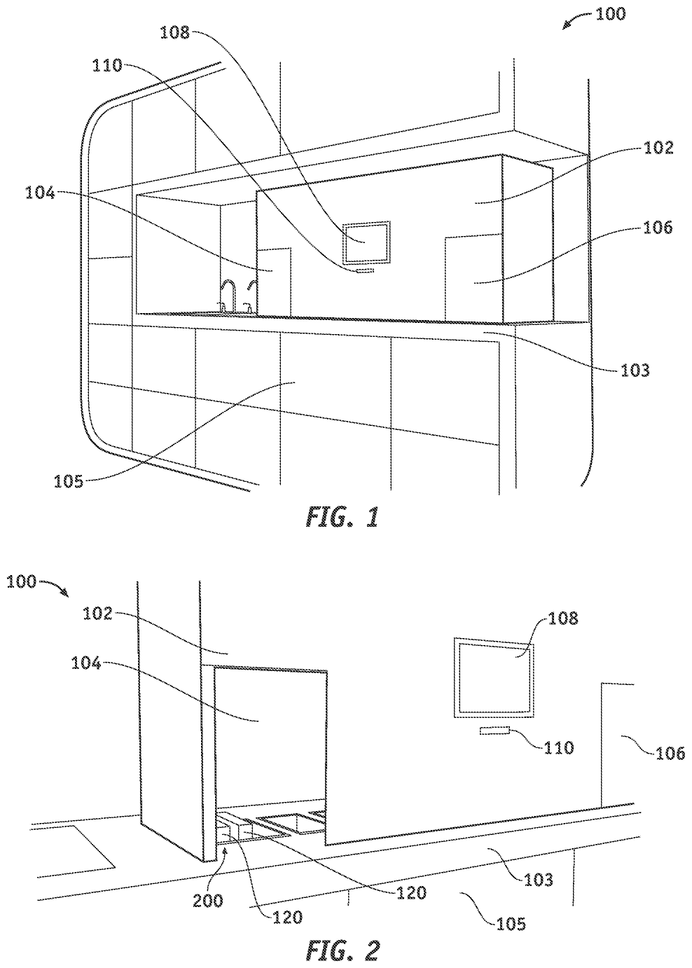

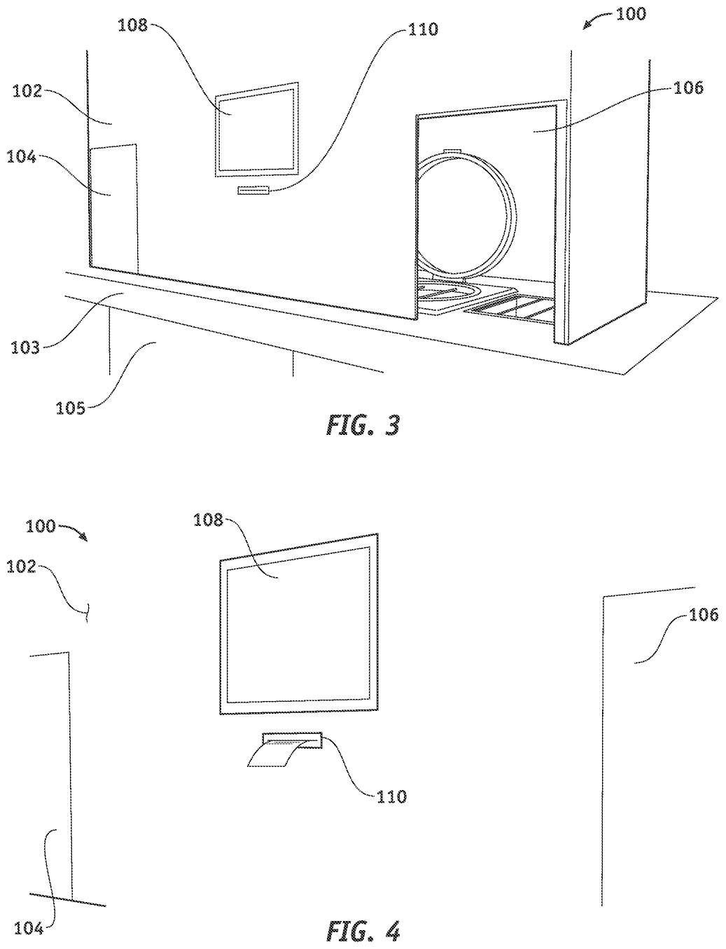

FIGS. 1-3 illustrate the inventive apparatus 100 in accordance with an embodiment of the invention. In particular, FIGS. 1-3 illustrate an apparatus 100 for reprocessing medical instruments, including medical instruments used in a hospital setting, medical instruments used in a doctor's office, dental instruments, or the like. The apparatus 100 is about the size of a standard cabinet unit that can typically be found in a doctor's office or dentist's office. For example, the apparatus 100 may be about 8-12 feet wide, 6-8 feet high, and 2-3 feet deep. Although FIG. 1 depicts the apparatus 100 having a sink station, one of ordinary skill in the art would readily appreciate that the sink station is optional and that an apparatus without the sink station would be a more compact apparatus.

The medical instrument reprocessing apparatus 100 includes an upper enclosure 102 having an entrance door 104 and an exit door 106. The enclosure 102 is small enough to fit on top of a counter 103. In particular, the enclosure 102 may be about 3-6 feet wide, about 3-6 feet high, and about 2-3 feet deep. A lower enclosure 105 conceals the bottom portion of the apparatus 100. The entrance door 104 provides access to the inside of the upper enclosure 102 so that a user can place dirty, contaminated instruments in the apparatus 100 through the entrance door 104. As explained in further detail below, there are several stations inside the enclosure 102, and the dirty instruments are processed through each station to clean, dry, package, and/or sterilize the instruments. After the instruments have been processed, a user can retrieve the clean, sterilized instruments from the enclosure 102 via the exit door 106. The entrance door 104 and the exit door 106 may be opened and closed manually or may open and close automatically or with the push of a button.

As shown in FIGS. 1-4, the apparatus 100 further includes a display 108 and a printer 110. The display 108 may be a monitor, touch screen, or the like, and may include, or be coupled to, a user input device that allows the user to input information and/or select a procedure before placing the dirty instruments in the enclosure 102. The display 108 may be configured to display desired information to the user, such as a status, time remaining in the cleaning and sterilizing procedures, or the like. The printer 110 is preferably configured to print out information regarding the cleaning and sterilizing procedure for a particular instrument cassette when the cassette is retrieved from the enclosure 102. The display 108 and printer 110 are optional. For example, in another embodiment, the apparatus 100 does not include the display 108 and printer 110, but rather the apparatus 100 is pre-programmed with a pre-determined cleaning and sterilizing procedure that is performed on each cassette that is processed through the apparatus 100. In another embodiment, the cleaning and sterilizing procedure may be selected through a mobile application or another remote device that is in communication with the apparatus 100, thus eliminating the need for the display 108 and printer 110.

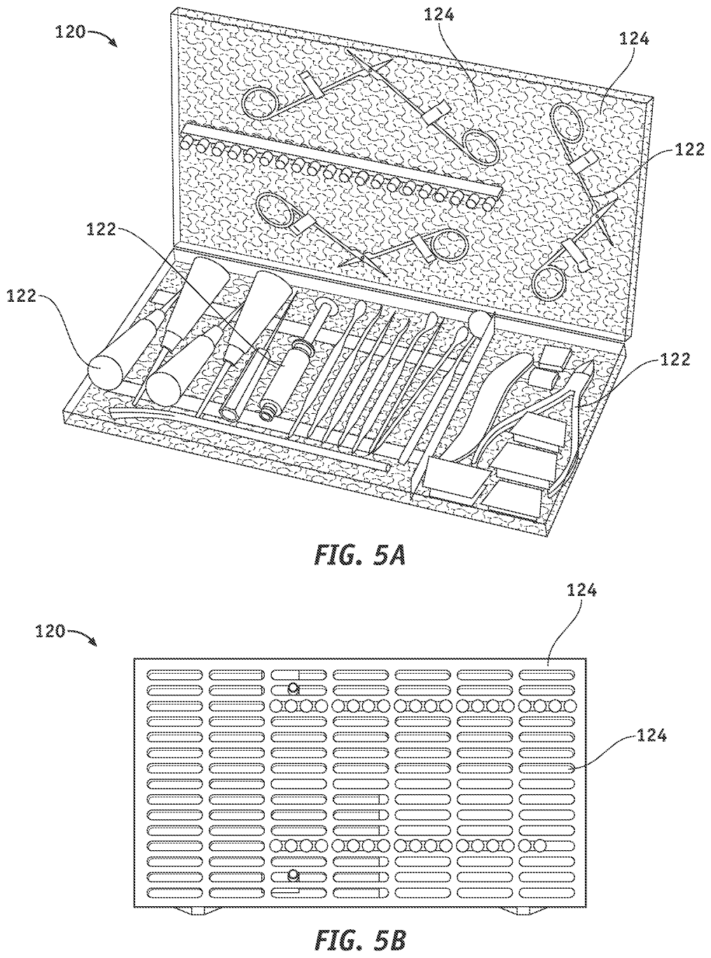

Before the dirty, contaminated instruments are placed inside the enclosure 102, the instruments are packed in a cassette 120, such as those depicted in FIGS. 5A and 5B. Instrument sterilization cassettes 120 typically include customized slots for the placement of various instruments 122. In this manner, the instruments 122 are held in place and are positioned to avoid touching each other during cleaning and sterilizing. A user positions the instruments 122 in the cassette 120, with each instrument 122 placed in a cassette slot that is specifically intended for that instrument. The cassette 120 has holes 124 that are large enough to freely pass fluids for cleaning and sterilizing so that essentially all of the surfaces of the instruments 122 disposed inside the cassette 120 are exposed to the fluids for effective cleaning and sterilizing. After packing the instruments 122 in the cassette 120, the cassette 120 with the dirty instruments therein is put into the enclosure 102 through the entrance door 104. Each cassette 120 may have attached thereto a unique barcode (not shown) for identifying the cassette 120 and keeping track of the cleaning and sterilizing procedures that are performed on that cassette 120.

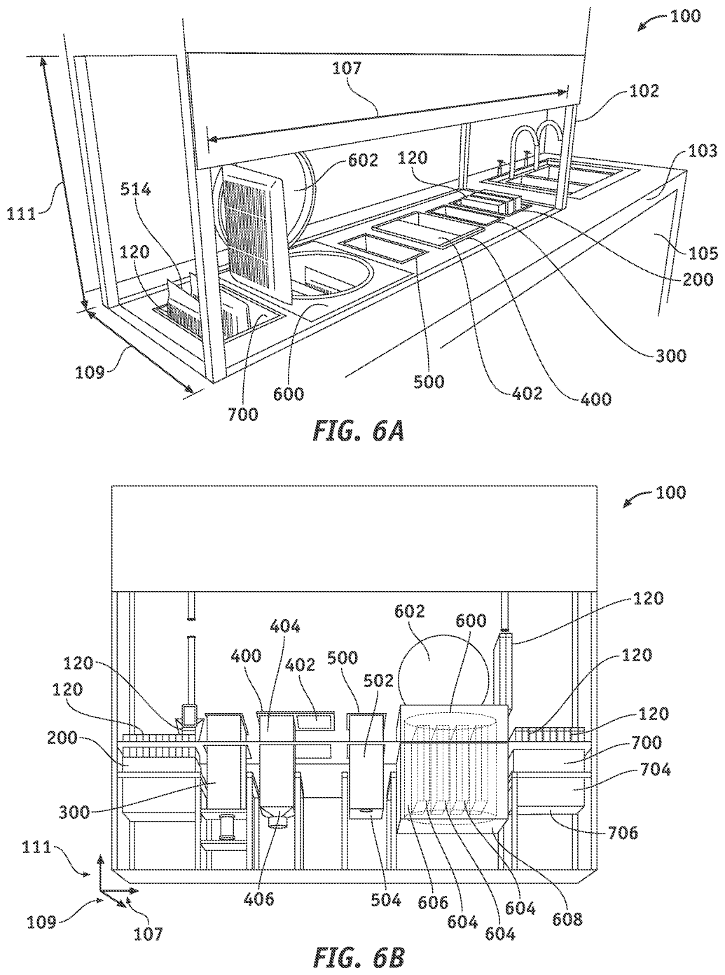

FIG. 6A depicts the apparatus 100 with the frame of the upper enclosure 102, but the walls of the upper enclosure 102 are removed to show the stations inside the enclosure 102. Similarly, FIG. 6B depicts the apparatus 100 with the walls of the upper enclosure 102 and the lower enclosure 105 removed to show the equipment that is disposed below the countertop 103. The stations are arranged in a linear row so that the instrument cassette 120 can easily be moved from one station to the next station down the line. That is, the instrument cassettes 120 are generally moved in the y direction 107 but are not generally moved in the x direction 109. The apparatus 100 includes a receiving area 200 adjacent to the entrance door 104. The receiving area 200 is also depicted in FIG. 2. After packing the dirty instruments in a cassette 120, the user positions the packed cassette 120 in the receiving area 200. In one embodiment, the apparatus 100 includes a fluid sprayer (not shown) coupled to a pre-cleaning solution reservoir (not shown) for spraying a pre-cleaning solution onto the cassettes 120 containing the dirty, contaminated instruments. The fluid sprayer is preferably positioned within, or adjacent to, the receiving area 200 so that the cassettes 120 can be sprayed with the pre-cleaning solution prior to being moved to the cleaning stations.

Next to the receiving area 200 is an ultrasound cleaning station 300. In one embodiment, the ultrasound cleaning station 300 is configured to turn on automatically when a cassette 120 is positioned in the ultrasound cleaning station 300. A water source is coupled to the ultrasound cleaning station 300, and a filter may be positioned between the water source and the ultrasound cleaning station 300 in order to filter the water that enters the ultrasound cleaning station 300. The ultrasound cleaning station 300 may include a cover or door (not shown) that may open and close automatically so that the ultrasound cleaning station 300 can be closed while cleaning is taking place and can be open during non-operating times. In this manner, the ultrasound cleaning station 300 can be open in order to position a cassette 120 therein, or remove a cassette 120 therefrom, and can be closed during ultrasonic cleaning of the cassette 120. The ultrasound cleaning station 300 is optional.

Next to the ultrasound cleaning station 300 is a jet washing and drying station 400. The jet washing and drying station 400 preferably includes a door 402 that closes during washing and drying and opens when a cassette 120 is being added to, or removed from, the jet washing and drying station 400. The door 402 on the jet washing and drying station 400 may be configured to open and close automatically. The jet washing and drying station 400 is configured to power wash the cassette 120 disposed therein at an elevated pressure. For example, the pressure wash may be at a pressure of about 500-1300 Psi. The jet washing and drying station 400 is further configured to dry the cassette 120 after the power washing.

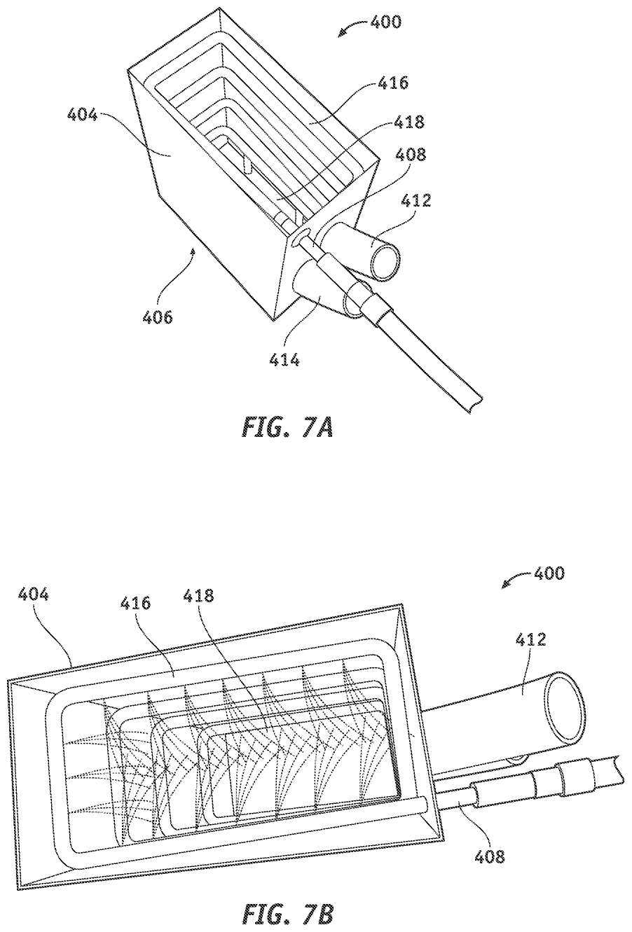

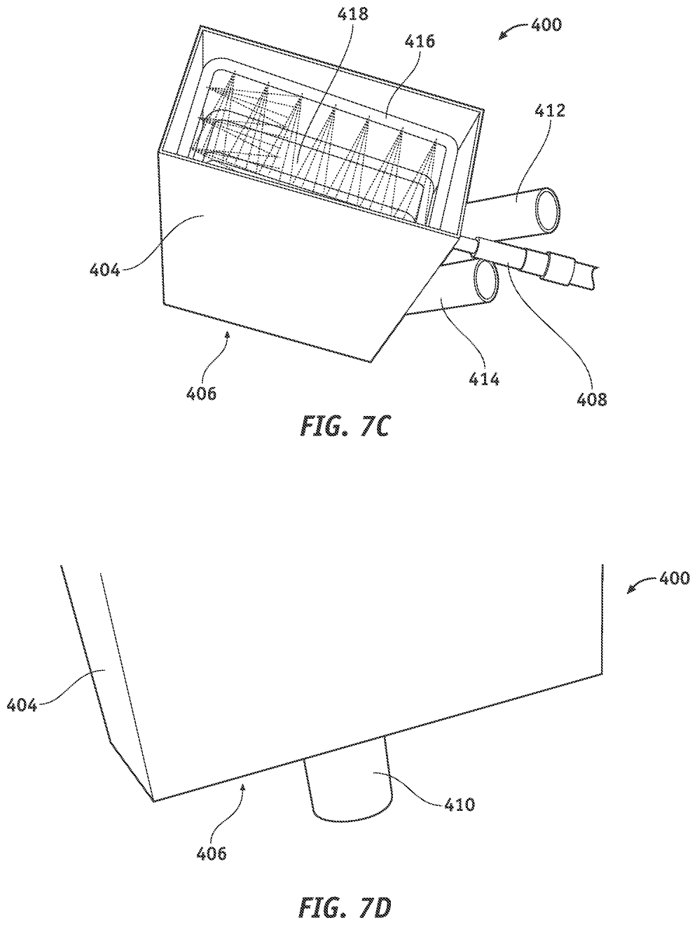

More details of one embodiment of the jet washing and drying station 400 are described with reference to FIGS. 7A-7D. The jet washing and drying station 400 includes sidewalls 404 and a bottom surface 406. The jet washing and drying station 400 further includes a liquid inlet 408, liquid outlet 410 (shown in FIG. 7D), air inlet 412, and air outlet 414. The liquid inlet 408 is coupled to jet wash sprayers 416 that are disposed throughout the interior of the jet washing and drying station 400 in order to wash the entire length and width of a cassette 120 disposed in the station 400. The sprayers 416 surround the cassette 120 when the cassette 120 is positioned in the slot 418 in the jet washing and drying station 400. The sprayers 416 are configured as power washers so that the cleaning fluid coming from the sprayers 416 is at an elevated pressure, such as 500-1300 Psi. Even though the embodiment depicted in FIGS. 7A-7D includes only one slot 418 for holding one cassette, it would be readily apparent to one of ordinary skill in the art that the jet wash and drying station may include two, three, or more slots for holding cassettes.

The jet washing and drying station depicted in FIGS. 7A-7D is merely an example of a jet washing and drying station that could be included in the apparatus. One of ordinary skill in the art would readily understand that other jet washing and drying configurations could be used without departing from the spirit and scope of the invention. For example, the jet washing and drying station may include rotating sprayer arms or sprayers that move up and down the length and/or width of the cassette. In another embodiment, the jet washing and drying are two separate stations. In other words, the apparatus includes a jet washing station and a drying station adjacent to the jet washing station.

In one embodiment, the apparatus 100 includes a camera and processor (not shown) that are configured to automatically determine whether the instruments in the cassette 120 are clean. For example, the camera is configured to capture images of the instruments after the cleaning procedure, and the processor is configured to process the images to determine whether the instruments are clean. The processor may include artificial intelligence, or machine learning, capabilities for determining whether the instruments are free of debris. The camera and processor are preferably positioned within, or adjacent to, the jet washing and drying station 400. In this manner, the apparatus 100 is able to automatically confirm that the instruments are free of debris before the instruments are packaged and/or sterilized.

Next to the jet washing and drying station 400 is a packaging station 500, as shown in FIGS. 6A and 6B. In one embodiment, the packaging station 500 includes a roll of packaging material that is configured to roll a pre-determined amount of packaging material into the packaging station 500. The packaging station 500 further includes an air blower configured for opening the package before a cassette 120 is positioned therein, and a heat sealer configured for sealing the package after the cassette 120 has been positioned therein. The packaging material may include a layer of paper and a layer of plastic that melts to seal the package when the heat sealer is applied thereto.

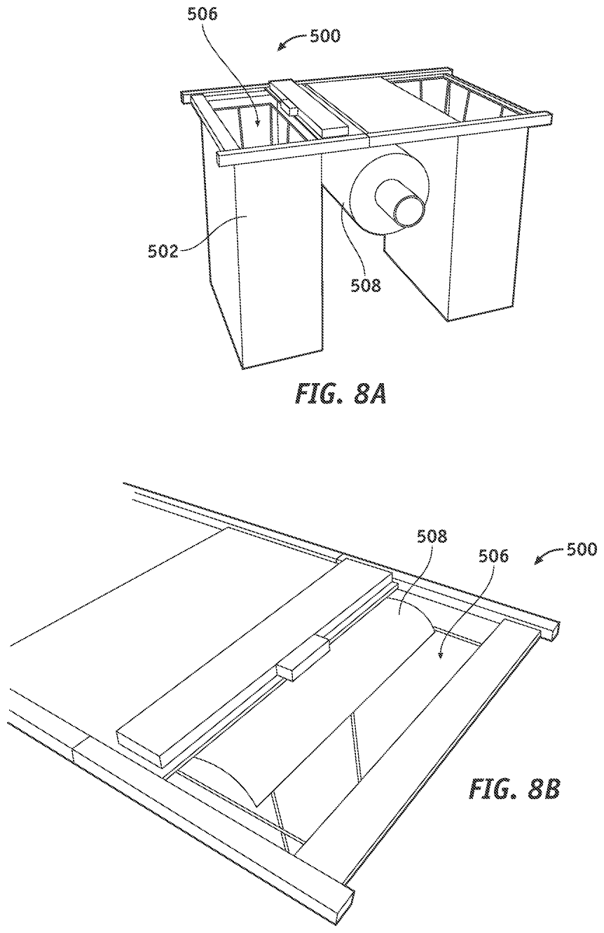

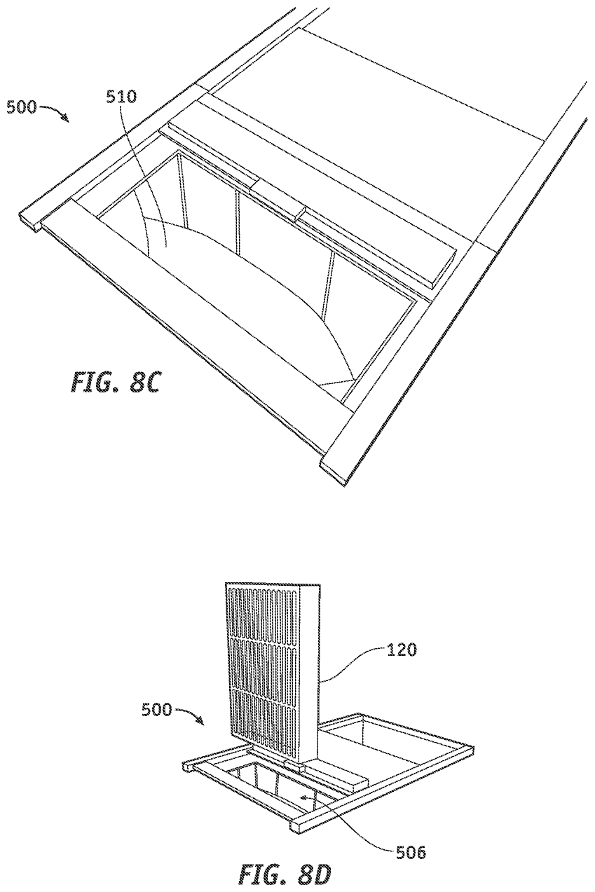

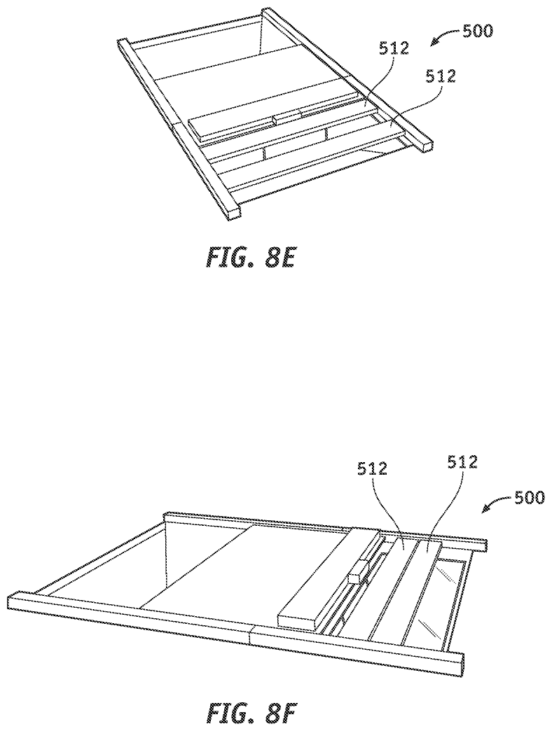

The packaging station 500 is described in more detail with reference to FIGS. 8A-8G. The packaging station 500 includes sidewalls 502 and a bottom surface 504 (also see FIG. 6B). Within the packaging station 500 is a slot 506 that is sized and shaped to hold a single instrument cassette 120. A roll of packing material 508 is adjacent to, and disposed outside of, the sidewalls 502. During use, the packaging material 508 rolls into the slot 506, as shown in FIG. 8B. Next, the desired amount of packaging material is cut off of the roll 508 and an air blower or other mechanical device is used to open the packaging material to form a pouch 510 that is disposed inside the packaging station 500. The pouch 510 is depicted in FIG. 8C. An instrument cassette 120 is deposited into the pouch 510, as shown in FIG. 8D, and a heat sealer is applied to the pouch 510 to seal the package around the cassette 120. FIG. 8E depicts the heat sealer 512 as it starts to close and FIG. 8F depicts the heat sealer 512 in the closed position in which the package is heat sealed. Next, as shown in FIG. 8G, the instrument cassette 120 enclosed in the sealed package 514 is removed from the packaging station 500.

The invention is not limited to the packaging station depicted in FIGS. 8A-8G. Rather, any kind of packaging station may be used to hermetically seal the instrument cassette 120 in packaging material that can withstand autoclaving and can protect the cleaned instruments within the cassette 120 from contamination. For example, the packaging station may include articulating arms that move around the cassette 120 to wrap up the cassette in sterilization-compatible wrapping material. In another embodiment, the packaging station may be equipped with self-sealing sterilization pouches, thus eliminating the need for the heat sealer.

Referring back to FIGS. 6A and 6B, the apparatus 100 further includes an autoclave station 600 having a lid 602 that closes and seals shut during the autoclave procedure. After the autoclave procedure, the lid 602 can be opened to provide access to the sterilized cassette 120. The autoclave station lid 602 may be configured to open and close automatically. The autoclave station 600 preferably includes a plurality of slots 604 for holding a plurality of cassettes 120 so that several cassettes 120 can be autoclaved at the same time. That is, each one of the slots 604 in the autoclave station 600 is sized and shaped to hold only one cassette 120 so that the cassettes 120 can be positioned away from the sidewalls 606 and bottom surface 608 of the autoclave station 600 and in a spaced apart position relative to each other. In this manner, over-crowding is avoided and the instruments inside the cassettes 120 are properly and efficiently sterilized.

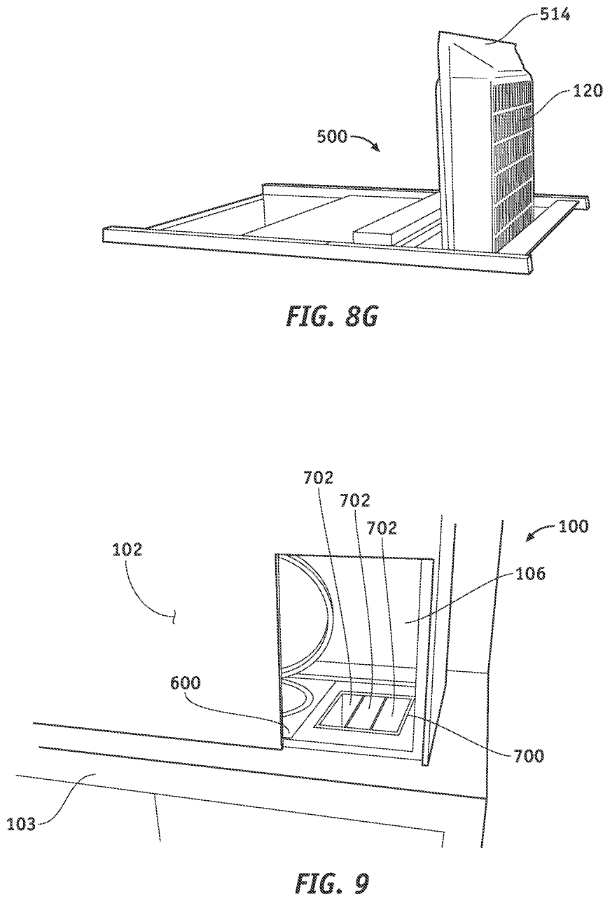

The apparatus 100 further includes a holding area 700. FIG. 6A further depicts the cassettes 120 after they have been cleaned, dried, packaged, sterilized and positioned in the holding area 700 awaiting removal from the enclosure 102. The cassettes 120 are enclosed in packaging material 514 by the time they arrive in the holding area 700. In one embodiment, as shown in FIG. 9, the holding area 700 includes a plurality of slots 702 for holding the cassettes 120. Each one of the slots 702 is sized and shaped for holding a single cassette 120. By positioning the cassettes 120 in the slots 702, the cassettes 120 are positioned in a spaced apart manner relative to each other and are held away from the sidewalls 704 and bottom surface 706 (see FIG. 6B) of the holding area 700.

Indeed, as shown in FIG. 6B, each one of the stations includes sidewalls and a bottom surface and at least one slot for preventing contact between the cassette 120 and the sidewalls and bottom surfaces. The one or more slots in each station are sized and shaped for holding a single cassette 120. The slots are made of a wire frame, or other similar construction that provides access to most, if not all, of the surfaces of the cassettes 120 and the medical instruments therein. Thus, essentially all of the instrument surfaces are exposed to the fluids in each station. This is especially important in the cleaning and sterilizing stations where it is necessary for all of the surfaces of the cassettes 120 and the instruments therein to be exposed to the fluids used in the cleaning and sterilizing procedures. It will be readily understood by one of ordinary skill in the art that each station includes at least one slot, but may include more than one slot. For example, as shown in FIG. 6B, the ultrasound cleaning station 300, jet washing and drying station 400, and packaging station 500 each include only one slot. However, it is within the scope of this invention for each of those stations to include two or more slots so that two or more cassettes 120 can be processed through those stations simultaneously. The receiving area 200, autoclave station 600, and holding area 700 are each depicted as having a plurality of slots, but it will be readily apparent to one of ordinary skill in the art that each of these stations or areas may have any number of slots, including only one slot. The number of cassette slots in each station can be customized, depending on size restrictions and work load requirements of the apparatus 100.

It should be readily understood by one of ordinary skill in the art that the apparatus 100 may be customized with more or fewer stations, depending on the needs of the user. For example, the user may want to eliminate the ultrasound cleaning station 300, add an additional jet washing and drying station 400, add an additional packaging station 500, or the like. Similarly, the receiving area 200 and holding area 700 may be eliminated for a more compact apparatus.

In one embodiment, the apparatus for reprocessing medical instruments includes only one station. For example, the apparatus may include only the jet washing and drying station. Similar to the embodiments described above, this embodiment may include an enclosure disposed on top of a counter, and a jet washing and drying station inside the enclosure. As discussed above, the jet washing and drying station has power washing capability. As such, the jet washing and drying station is quicker, more efficient, and more effective than conventional washer/disinfectors that are currently used to process medical devices. In other embodiments, the one or more stations may be arranged in a different configurations as would be apparent to a person of ordinary skill in the art without departing from the scope of the invention.

A cassette 120 containing instruments is automatically moved from one station to the next. For example, the apparatus may include a robotic arm that picks up the cassette 120 and moves the cassette 120 down the line to the next station. The distal end of the robotic arm includes a mechanism for holding and lifting the cassette 120 while transporting the cassette 120. For example, the robotic arm may include a gripper, grabber, claw, magnetic mechanism, electromagnetic mechanism, or the like. In other embodiments, the cassette 120 is moved from one station to the next by a conveyor belt, actuator, or other motorized transport mechanism. In yet another embodiment, the cassette 120 is moved from one station to the next manually, which requires a user to have access to the upper enclosure 102 and move the cassette 120 from one station to the next station.

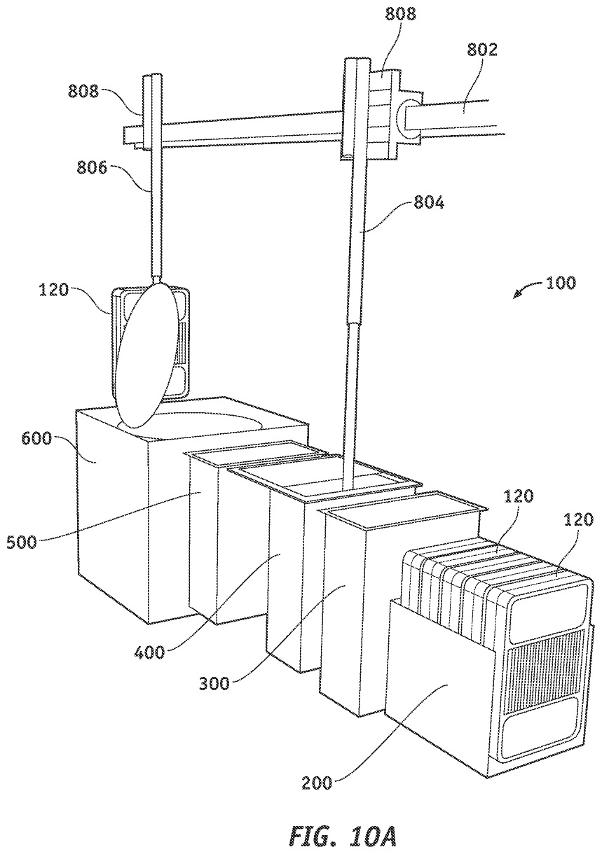

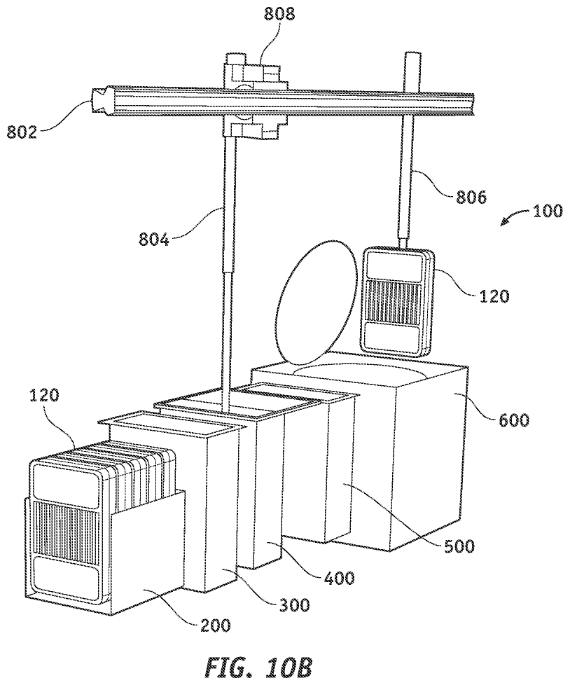

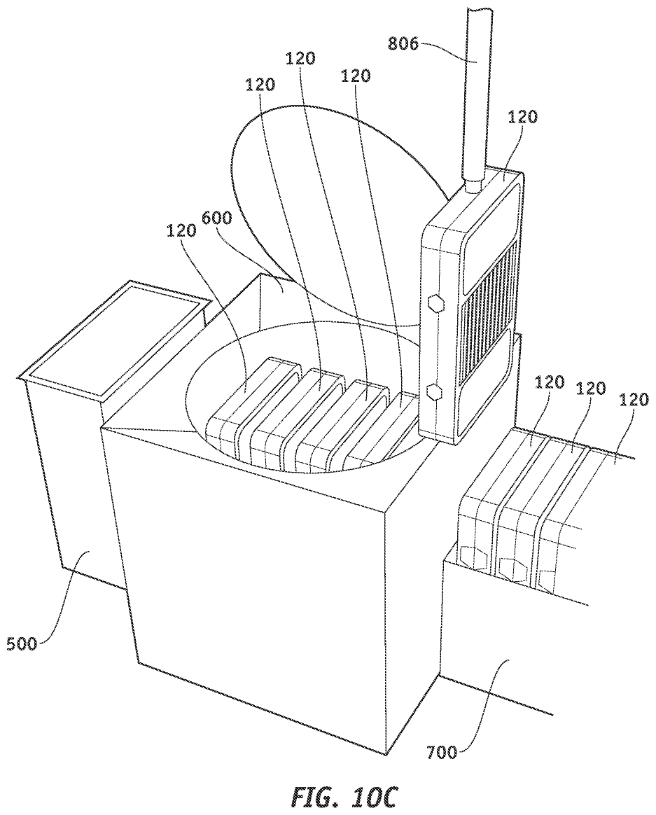

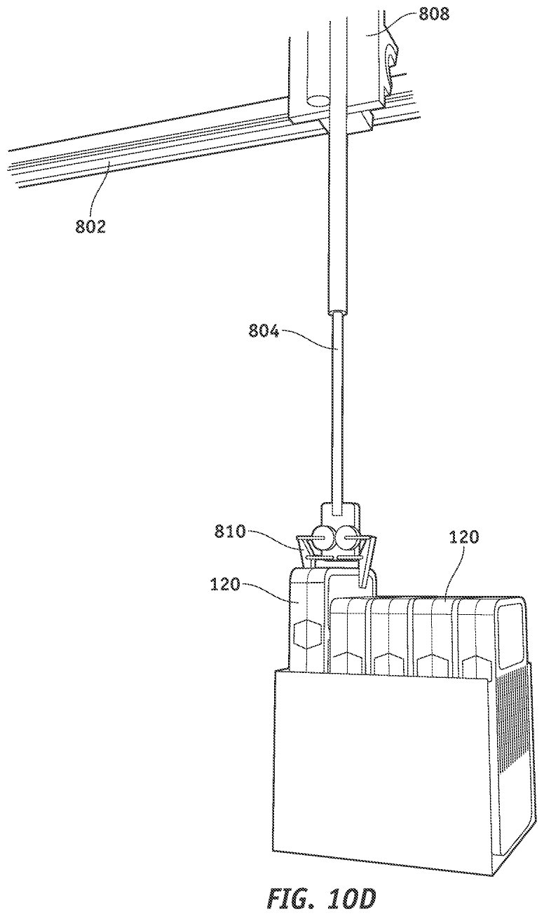

One embodiment of the apparatus 100 including a robotic arm mechanism is depicted in FIGS. 10A-10D. As shown in FIGS. 10A and 10B, the apparatus 100 includes an overhead track 802 that is linearly positioned above the linear row of stations. The apparatus 100 further includes two separate robotic arms 804 and 806 that are configured to traverse linearly along the track 802. The robotic arms 804 and 806 are coupled to the track 802 through a programmable, motorized bracket assembly 808 that is configured to traverse linearly along the track 802. When the robotic arms 804 and 806 are in a desired linear position, the robotic arms 804, 806 extend downwards towards the station to grasp a cassette 120 or position a cassette 120 in the station. That is, the robotic arms 804, 806 are configured to move in the y direction 107 (along the track 802) and the z direction 111 (up and down), but not in the x direction 109 (see FIGS. 6A and 6B). For example, FIGS. 10A and 10B depict one of the robotic arms 804 in a downwardly extended position, and the other robotic arm 806 in a retracted position. One advantage to having two robotic arms 804, 806 is that one of the robotic arms 804, 806 may be designated for handling cassettes 120 containing dirty, contaminated instruments, while the other one of the robotic arms 804, 806 may be designated for handling cassettes 120 that have been through a cleaning or sterilizing cycle. Another advantage to having two separate robotic arms 804, 806 is that both of the arms 804, 806 can simultaneously be moving cassettes 120 between stations in the apparatus 100, which may speed up the procedure. FIG. 10C depicts one of the robotic arms 806 moving a cassette 120 between the autoclave station 600 and the holding area 700. As shown in FIG. 10C, the autoclave station 600 includes four slots for holding four cassettes 120 in a spaced apart configuration. In this manner, the cassettes 120 are not in contact with one another during the autoclaving procedure, and autoclave over-crowding is avoided.

The distal ends of the robotic arms 804, 806 are equipped with grabbers, graspers, or claws 810 (shown in FIG. 10D) that open and close to release and grab the cassettes 120. FIG. 10D depicts the grabber 810 on the distal end of one of the robotic arms 804. The grabber 810 is designed to automatically open and close and to grab, lift, transport, lower, and release the cassettes 120.

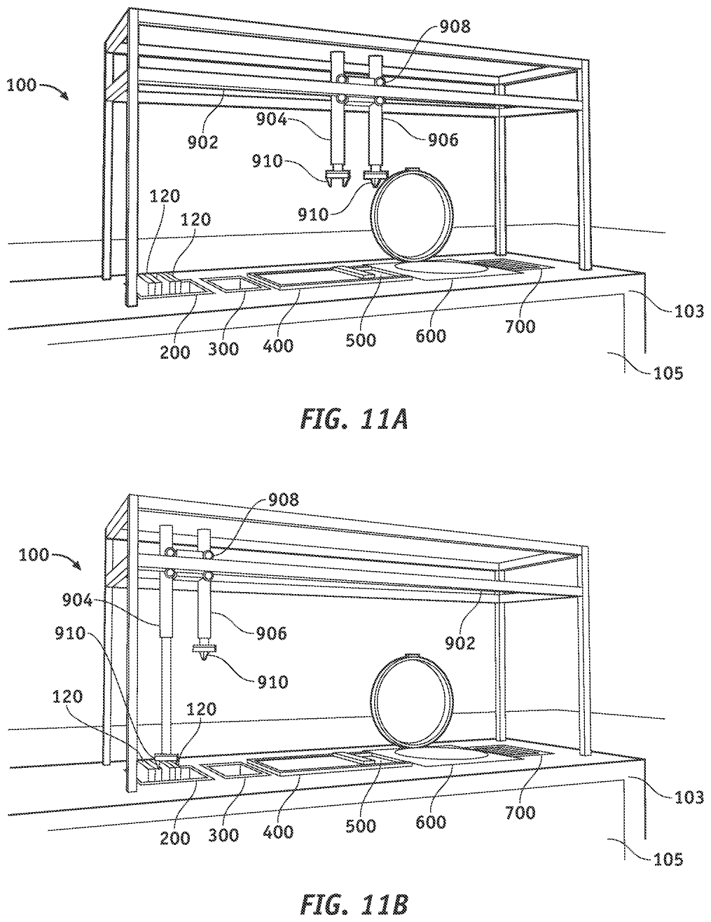

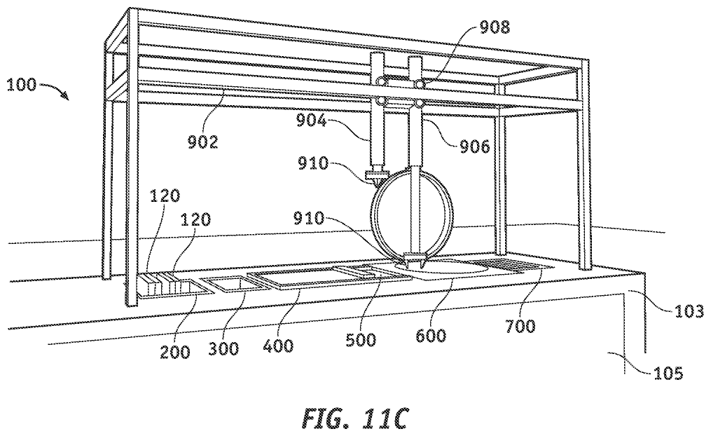

In another embodiment, depicted in FIGS. 11A-11C, the apparatus 100 may include two robotic arms 904, 906 that are coupled together. That is, the robotic arms 904, 906 are coupled to the overhead track 902 through a single, wheeled track traversal apparatus 908. Even though the robotic arms 904, 906 are simultaneously moved along the track 902 in fixed positions relative to each other, the robotic arms 904, 906 are preferably configured to move in the z-direction 111 independently of each other. Each of the robotic arms 904, 906 includes a grabber or a claw 910 configured to grab a cassette 120. As shown in FIG. 11A, the grabber 910 on one of the robotic arms 904 is open, while the grabber 910 on the other robotic arm 906 is closed. As shown in FIG. 11B, one of the robotic arms 904 is extended downwardly to grasp and remove a cassette 120 from the receiving area 200, while the other robotic arm 906 remains in the retracted position. As shown in FIG. 11C, one of the robotic arms 906 is extended downwardly towards the autoclave station 600 to either grasp or release a cassette 120, while the other robotic arm 904 remains in the retracted position. Similar to the previous embodiment, it is advantageous for the apparatus 100 to include two robotic arms 904, 906 so that one of the arms can be designated for interacting with the cassettes 120 containing the dirty, contaminated instruments, while the other arm can be designated for interacting with the cassettes 120 that have undergone a cleaning and/or sterilizing procedure, thereby avoiding cross-contamination of the cassettes 120. Another advantage of the embodiment that includes two robotic arms is that both robotic arms can be utilized simultaneously to move cassettes 120 through the stations, which may reduce the time it takes to clean, package, and sterilize the cassettes 120 and the instruments therein.

In yet another embodiment (not shown), the apparatus may include a single robotic arm having one or two grabbers coupled to the distal end of the robotic arm. One of the grabbers may be designated for handling the cassettes 120 as they enter the apparatus, and the other grabber may be designated for handling the cassettes 120 that have been cleaned and/or sanitized. As such, cross-contamination of the cassettes 120 can be avoided.

Still further, in another embodiment, the apparatus 100 includes a UV light (not shown) disposed inside the enclosure. The UV light may be configured to sterilize the inside of the enclosure 102. For example, during a time when the apparatus 100 is not in use (such as at night when the dental or medical office is closed), the UV light may be activated to clean the inside of the enclosure 102.

In yet another embodiment, the apparatus 100 is configured to automatically perform spore tests to confirm sterilization per CDC guidelines.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and an apparatus for reprocessing medical devices through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. An apparatus for processing medical instruments, the apparatus comprising:
an enclosure having an entrance door and an exit door, the enclosure arranged so that a cassette containing dirty instruments is deposited into the enclosure through the entrance door, the dirty instruments are cleaned, packaged, and sterilized inside the enclosure, and the clean, packaged, sterilized instruments are removed from the enclosure through the exit door, wherein the enclosure comprises a plurality of stations therein, the plurality of stations comprising:
a jet washing and drying station;
a packaging station; and
an autoclave station,
wherein each station comprises sidewalls and a bottom surface,
wherein each station comprises at least one slot that is sized and shaped to hold only one cassette, and
wherein each slot in each station comprises a wire frame so that the cassette, when positioned in the slot, does not contact the sidewalls or bottom surface of the station.

2. The apparatus of claim 1, further comprising an ultrasound cleaning station.

3. The apparatus of claim 2, wherein the ultrasound cleaning station comprises a door that is configured to be closed during cleaning, wherein the door opens to provide access to the ultrasound cleaning station for depositing the cassette into, or removing the cassette from, the ultrasound cleaning station.

4. The apparatus of claim 3, wherein the ultrasound cleaning station door is configured to open and close automatically.

5. The apparatus of claim 2, wherein the ultrasound cleaning station is configured to automatically turn on when the cassette is positioned in the ultrasound cleaning station.

6. The apparatus of claim 2, further comprising an instrument cleaning solution reservoir coupled to the ultrasound cleaning station.

7. The apparatus of claim 2, wherein the ultrasound cleaning station is configured for being coupled to a water source, and wherein the apparatus further comprises a filter for filtering water that enters the ultrasound cleaning station.

8. The apparatus of claim 1, further comprising a holding area in the enclosure, wherein the holding area is adjacent to the exit door.

9. The apparatus of claim 1, further comprising a receiving area in the enclosure, wherein the receiving area is positioned adjacent to the entrance door so that a user has access to the receiving area through the entrance door in order to position the instrument cassette through the entrance door an into the receiving area.

10. The apparatus of claim 9, further comprising a fluid sprayer adapted to be coupled to a pre-cleaning solution for spraying the pre-cleaning solution on the external surfaces of the dirty instruments.

11. The apparatus of claim 10, wherein the fluid sprayer is within, or adjacent to, the receiving area.

12. The apparatus of claim 1, wherein the entrance door and exit door open and close automatically.

13. The apparatus of claim 1, further comprising a robotic arm in the enclosure.

14. The apparatus of claim 13, wherein the robotic arm is coupled to a linear track positioned above the stations in the enclosure.

15. The apparatus of claim 14, wherein the robotic arm is configured to traverse the linear track in the y-direction.

16. The apparatus of claim 15, wherein the robotic arm is configured to move in the z-direction towards and away from the stations in the enclosure.

17. The apparatus of claim 16, wherein the robotic arm comprises a gripper that closes and opens for grasping and releasing the cassettes.

18. The apparatus of claim 17, further comprising a second gripper attached to the robotic arm.

19. The apparatus of claim 1, further comprising two robotic arms in the enclosure, wherein a first robotic arm is configured for engaging with the cassette containing dirty instruments, and a second robotic arm is configured for engaging with the cassette after the instruments have been cleaned.

20. The apparatus of claim 1, further comprising a camera and a processor configured for automatically determining whether the medical instruments are clean.

21. The apparatus of claim 20, wherein the camera and processor are positioned adjacent to the jet washing and drying station in order to determine whether the instruments are clean prior to packaging.

22. The apparatus of claim 20, wherein the processor comprises artificial intelligence capability for determining whether the instruments are clean.

23. The apparatus of claim 1, wherein the stations are arranged in a linear row.

24. The apparatus of claim 23, wherein the stations are arranged in the linear row so that:
the jet washing and drying station is adjacent to the packaging station; and
the packaging station is disposed between the jet washing and drying station and the autoclave station.

25. The apparatus of claim 1, further comprising a UV light in the enclosure, the UV light adapted to sterilize an inside of the enclosure.

26. The apparatus of claim 1, wherein the jet washing and drying station comprises a door that is configured to be closed during washing and drying, wherein the door is further configured to be open before and after the washing and drying.

27. The apparatus of claim 26, wherein the jet washing and drying station door is configured to open and close automatically.

28. The apparatus of claim 1, wherein the autoclave station comprises a lid that closes to seal the autoclave station closed during operation of the autoclave station.

29. The apparatus of claim 28, wherein the autoclave station lid is configured to open and close automatically.

30. The apparatus of claim 1, wherein each station comprises at least two slots, each slot being sized and shaped to hold only one cassette.

31. The apparatus of claim 30, wherein each station comprises at least three slots, each slot being sized and shaped to hold only one cassette.

32. The apparatus of claim 1, wherein the packaging station comprises a roller of packaging material, an air blower for opening the package before the cassette is positioned in the package, and a heat sealer for sealing the package after the cassette is positioned therein.

33. An apparatus for processing medical instruments, the apparatus comprising:
an enclosure having an entrance door and an exit door, the enclosure arranged so that a cassette containing dirty instruments is deposited into the enclosure through the entrance door, the dirty instruments are cleaned, sterilized, and packaged inside the enclosure, and the clean, sterilized, packaged instruments are removed from the enclosure through the exit door, wherein the enclosure comprises a plurality of stations arranged in a linear row, wherein each station comprises:
sidewalls,
a bottom surface, and
at least one slot that is sized and shaped to hold only one cassette,
wherein each slot in each station comprises a wire frame configured to hold the cassette in a position that is spaced apart from the sidewalls and bottom surface of the station so that all sides of the instruments are fluidly accessible through the cassette and the wire frame.

34. The apparatus of claim 33, wherein the plurality of stations comprises:
an ultrasound cleaning station;
a jet washing and drying station;
a packaging station; and
an autoclave station.

35. The apparatus of claim 34, further comprising a receiving area adjacent to the entrance door and a holding area adjacent to the exit door.
